(19) United States
(12) Patent Application Publication
(10) Pub. No.: US 2010/0112569 A1
(43) Pub. Date: May 6, 2010

(54) MICROFLUIDIC DEVICES AND METHODS OF GENERATING AND USING SAME

(76) Inventors: Roy Bar-Ziv, Rechovot (IL); Amnon Buxboim, Zikhron-Yaakov (IL); Margherita Morpurgo, Padova (IT)

Correspondence Address: MARTIN D. MOYNIHAN d/b/a PRTSI, INC., P.O. BOX 16446, ARLINGTON, VA, 22215, US

(21) Appl. No.: 12/449,061

(22) PCT Filed: Jan. 23, 2008
(86) PCT No.: PCT/IL08/00107

§ 371 Date: Nov. 24, 2009

Related U.S. Application Data
(60) Provisional application No. 60/881,849, filed on Jan. 23, 2007.

(51) Int. Cl. C12Q 1/68; G01N 33/00; C12M 1/34; B05D 1/40

(52) U.S. Cl. 435/6; 422/68.1; 435/287.2; 427/331

(57) ABSTRACT

A microfluidic device comprising a substrate having formed therein microfluidic paths, at least a portion of the microfluidic paths having attached thereto a plurality of monolayers, wherein at least a portion of the monolayers comprises a photoactivatable group capable of generating a reactive group upon exposure to a light source, the reactive group being for binding a screenable moiety.

### FIELD AND BACKGROUND OF THE INVENTION

The present invention relates to microfluidic devices and, more particularly, to methods of generating and using microfluidic devices.

Microfluidics has emerged as a revolutionizing technology for a “lab-on-a-chip” with numerous applications. The perspectives have recently been described in a series of review articles [see for example, Craighead, H. Future lab-on-a-chip technologies for interrogating individual molecules. Nature 442, 387-393 (2006); deMello, A. J. Control and detection of chemical reactions in microfluidic systems. Nature 442, 394-402 (2006); El-Ali, J., Sorger, P. K. & Jensen, K. F. Cells on chips. Nature 442, 403-411 (2006); Janasek, D., Franzke, J. & Manz, A. Scaling and the design of miniaturized chemical-analysis systems. Nature 442, 374-380 (2006); Psaltis, D., Quake, S. R. & Yang, C. H. Developing optofluidic technology through the fusion of microfluidics and optics. Nature 442, 381-386 (2006); Whitesides, G. M. The origins and the future of microfluidics. Nature 442, 368-373 (2006); Yager, P. et al. Microfluidic diagnostic technologies for global public health. Nature 442, 412-418 (2006)].

Microfluidic (MF) devices are commonly made of channels and compartments/chambers carved in a transparent elastomer (commonly: poly-di-methyl-siloxane; PDMS) that is sealed with a glass slide. Fluids carrying reagents, cells, biomolecules, and the like, are introduced into the device by thin tubing that connects the minute (nano-liter) volumes of the channels to external macroscopic reservoirs. Tight sealing of MF devices with glass slides is essential for their function as the forces applied on the elastomer by the external fluid pressure sources are strong enough to crack open the boundaries, leading to leak of fluids. As a result, the MF channels and glass surfaces are homogenously treated (oxidation) to ensure tight and irreversible sealing before any fluids are introduced in the device. This treatment is quite harsh and would likely damage any predetermined pattern of biomolecules on the glass slide.

Biochips are inherently relevant for MF devices since immobilization of molecules and on-chip spatial definition can facilitate greater flexibility in design of various devices where fluids in contact with the chip are transported from one location to another. This relates to biochemical reactions on the chip, for example, DNA hybridization, enzyme catalysis and protein biosynthesis, as well as for adhesion of cells. The biochip methodologies are generally divided into two: One, mechanical spotting of molecules at designated regions followed by washing of excess unbound material. Two, immobilization of molecules by a light-directed approach (UV photolithography) on a photosensitive layer with all steps carried out by flow of reagents over surface exposed regions without physical manipulation. One such approach is described in PCT Application No. WO2006/064505 to the present inventor.

The vast majority of applications use the easier approach first since the light-directed method is currently not widespread due to its demanding technical expertise. However it is evident that the light-directed approach best fits MF since it requires no physical contact with the surfaces. In principle, this is easy: a glass slide is coated with the photosensitive layer, sealed against the MF channels, which renders a device ready for immobilization of biomolecules on its floor. However, this fails since the surface treatment before sealing the channel will likely destroy the photosensitive layer on the glass. Another possibility would be to first seal the MF channel with glass and then coat the glass with the photosensitive layer. This also fails since the formation of photosensitive layers requires incubating the glass with organic solvents, which are not compatible with the MF elastomer.

There is thus a widely recognized need for, and it would be highly advantageous to have, microfluidic devices and methods of generating same which are devoid of the above limitations.

### SUMMARY OF THE INVENTION

According to one aspect of the present invention there is provided a microfluidic device comprising a substrate having formed therein microfluidic paths, at least a portion of the microfluidic paths having attached thereto a plurality of monolayers, wherein at least a portion of the monolayers comprises a photoactivatable group capable of generating a reactive group upon exposure to a light source, the reactive group being for binding a screenable moiety.

According to another aspect of the present invention there is provided a method of manufacturing a microfluidic device, the method comprising:

(a) providing a substrate having formed therein microfluidic paths; and

(b) coating the substrate with a compound having a functionalized group bound to the substrate, a photoactivatable group capable of generating a reactive group upon exposure to a light source, the reactive group being for binding a screenable moiety, and a polymer capable of forming a monolayer on the substrate and having the functionalized group and the photoactivatable group attached thereto, so as to form a plurality of monolayers of the polymer over the substrate, wherein the coating is restricted to an area so as to allow sealing of the microfluidic device while retaining functionality of the photoactivatable group.

According to still further features in the described preferred embodiments the method further comprises sealing the microfluidic device following step (b).

According to yet another aspect of the present invention there is provided a method of nucleic acid amplification, the method comprising:

(a) providing the microfluidic device;

(b) binding at least one nucleic acid template to the reactive group; and

(c) flowing at least one amplification reagent into reaction sites of the microfluidic device under conditions which allow nucleic acid amplification.

According to still further features in the described preferred embodiments the at least one amplification reagent is selected from the group consisting of primers, polymerase, nucleotides, metal ions, buffer and cofactors.

According to still further features in the described preferred embodiments the nucleic acid amplification is effected according to a method selected from the group consisting of a PCR, a ligase amplification reaction (LCR), a transcription amplification, a self-sustained sequence replication and a nucleic acid based sequence amplification (NASBA).

According to still further features in the described preferred embodiments the method further comprises detecting the amplified product of (c). According to still another aspect of the present invention there is provided a method of detecting a nucleic acid sequence of interest, the method comprising:

(a) providing the microfluidic device;

(b) binding at least one nucleic acid probe to the reactive group; and

(c) flowing a labeled probe into reaction sites of the microfluidic device under conditions under conditions allowing detection of the nucleic acid sequence of interest.

According to still further features in the described preferred embodiments the nucleic acid sequence of interest comprises a SNP.

According to an additional aspect of the present invention there is provided a method of identifying an agent having a desired activity, the method comprising:

(a) providing the microfluidic device;

(b) binding a plurality of agents to the reactive groups such that one agent is disposed in one reaction site at the plurality of areaction sites of the microfluidic device; and

(c) flowing reaction mixtures into reaction sites of the microfluidic device;

(d) monitofing the desired activity following the flowing thereby to thereby identify the agent having the desired activity.

According to still further features in the described preferred embodiments the monolayers being composed of a compound which comprises a general formula I:

X-L—Y Formula I

wherein:

X is a functionalized group capable of binding to the substrate;

L is a polymer capable of forming the monolayer onto the substrate; and

Y is the photoactivatable group capable of generating the reactive group upon exposure to the light.

According to still further features in the described preferred embodiments the functionalized group comprises at least one reactive silyl group.

According to still further features in the described preferred embodiments the reactive silyl group is selected from the group consisting of trialkoxysilane, alkyldialkoxysilane, alkoxydialkylsilane, trihalosilane, alkyldihalosilane and dialkylhalosilane.

According to still further features in the described preferred embodiments the reactive silyl group is trialkoxysilane.

According to still further features in the described preferred embodiments the functionalized group comprises an alkyl terminating with the trialkoxysilane.

According to still further features in the described preferred embodiments the functionalized group comprises at least one reactive silyl group selected from the group consisting of trialkoxysilane, alkyldialkoxysilane, alkoxydialkylsilane, trihalosilane, alkyldihalosilane and dialkylhalosilane.

According to still further features in the described preferred embodiments the polymer comprises a substituted or unsubstituted polyethylene glycol (PEG).

According to still further features in the described preferred embodiments the polyethylene glycol has a molecular weight that ranges from about 400 grams/mol and about 10000 grams/mol.

According to still further features in the described preferred embodiments the polyethylene glycol has a molecular weight that ranges from about 2000 grams/mol to about 5000 grams/mol.

According to still further features in the described preferred embodiments the polyethylene glycol has a general formula II:

—(CR¹R²CR³R⁴O)n- Formula II

wherein:

n is an integer from 10 to 200; and

R¹, R², R³ and R⁴ are each independently selected from the group consisting of hydrogen, alkyl, cycloalkyl, aryl, alkenyl alkynyl, alkoxy, thioalkoxy, aryloxy and thioaryloxy.

According to still further features in the described preferred embodiments R¹, R², R³ and R⁴ are each hydrogen.

According to still further features in the described preferred embodiments n is an integer from 60 to 100.

According to still further features in the described preferred embodiments the functionalized group is attached to the polymer via a linking moiety.

According to still further features in the described preferred embodiments the linking moiety is selected from the group consisting of oxygen, sulfur, amine, amide, carboxylate, carbamate, N-carbamate, sulphonate, sulphonamide, phosphate, hydrazine, hydrazide and derivatives thereof.

According to still further features in the described preferred embodiments the linking moiety is amide.

According to still further features in the described preferred embodiments the reactive group generated upon exposure of the photoactivatable group to the light is selected from the group consisting of amine, hydroxy, thiohydroxy, halo, alkoxy, thioalkoxy, aryloxy, thioaryloxy, carboxylate, phosphate, phosphonate, sulfate and sulfonate.

According to still further features in the described preferred embodiments the photoactivatable group comprises a carbamate.

According to still further features in the described preferred embodiments the reactive group generated upon exposure of the photoactivatable group to the light is amine.

According to still further features in the described preferred embodiments the photoactivatable group comprises a 6-nitroveratryl chloroformate residue, a 2-nitrobenzyl residue, a 2-nitroanilino residue, a phenacyl residue, a phenoxy residue, an azidoaryl residue, a sulfonic ester residue, a desyl residue, a p-hydroxyphenacyl residue, a 7-methoxy coumarin residue, a o-ethylacetophenone residue, a 3,5-dimethylphenacyl residue, a dimethyl dimethoxybenzyloxy residue, a 5-bromo-7-nitroindolinyl residue, a o-hydroxy-α-methyl cinnamoyl residue and a 2-oxymethylene anthraquinone residue.

According to still further features in the described preferred embodiments the light is selected from the group consisting of UV, IR, visible light and monochromatic light of a predetermined wavelength.

According to still further features in the described preferred embodiments the substrate comprises a silica-based material.

According to still further features in the described preferred embodiments the substrate comprises an elsatomeric material.

According to still further features in the described preferred embodiments the screenable moiety is selected from the group consisting of a screenable moiety and chemical moiety.

According to still further features in the described preferred embodiments the screenable moiety is selected from the group consisting of a peptide, a protein, a glycoprotein, a proteoglycan, a nucleic acid, an oligonucleotide, an antibody, a carbohydrate, a hormone, a steroid, a lipid, a cell, a microorganism, an enzyme, and a growth factor.

According to still further features in the described preferred embodiments the chemical moiety is selected from the group consisting of a chelating agent, an inhibitor, a substrate and a ligand.

According to still further features in the described preferred embodiments, the device further comprises at least one valve for regulating fluid infusion into the microfluidic paths.

According to still further features in the described preferred embodiments the microfluidic paths comprise microchannels and/or microchambers.

According to still further features in the described preferred embodiments the microchambers form reaction sites.

According to still further features in the described preferred embodiments the device further comprises at least one reagent inlet port.

According to still further features in the described preferred embodiments the device further comprises at least one reaction outlet port.

According to still further features in the described preferred embodiments the device further comprises at least one external reservoir being in fluid communication with the reagent inlet port.

The present invention successfully addresses the shortcomings of the presently known configurations by providing novel microfluidic devices and methods of generating and using same.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

General Terminology

As used herein, the term “amine” describes both a —NR′R″ group and a —NR′— group, wherein R′ and R″ are each independently hydrogen, alkyl, cycloalkyl, aryl, as these terms are defined herein.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

The term “amine” is used herein to describe a —NR′R″ group in cases where the amine is an end group, as defined hereinunder, and is used herein to describe a —NR′— group in cases where the amine is a linking group.

Herein throughout, the phrase “end group” describes a group (a substituent) that is attached to another moiety in the compound via one atom thereof.

The phrase “linking group” describes a group (a substituent) that is attached to another moiety in the compound via two or more atoms thereof.

The term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking group, as this phrase is defined hereinabove, connecting another moiety at each end thereof.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The alkyl group can be an end group, as this term is defined hereinabove, wherein it is attached to a single adjacent atom, or a linking group, as this term is defined hereinabove, connecting two or more moieties at two or more positions thereof.

The term “halide” or “halo” describes fluorine, chlorine, bromine or iodine.

The term “haloalkyl” describes an alkyl group as defined above, further substituted by one or more halide.

The term “sulfate” describes a —O—S(═O)₂—OR′ end group, or an —O—S(═O)₂—O— linking group, as these phrases are defined hereinabove, where R′ is as defined hereinabove. This term further encompasses thiosulfates.

The term “thiosulfate” describes a —O—S(═S)(═O)—OR′ end group or a —O—S(═S)(═O)—O— linking group, as these phrases are defined hereinabove, where R′ is as defined hereinabove.

The term “sulfonate” describes a —S(═O)₂—R′ end group or an —S(═O)₂— linking group, as these phrases are defined hereinabove, where R′ is as defined herein.

The term “sulfonamide” describes a —S(═O)₂—NR′R″ end group or a —S(═O)₂—NR′— linking group, as these phrases are defined hereinabove, with R′ and R″ as defined herein. This term encompasses the terms N-sulfonamide and S-sulfonamide.

The term “N-sulfonamide” describes an R′S(═O)₂—NR″— end group or a —S(═O)₂—NR′— linking group, as these phrases are defined hereinabove, where R′ and R″ are as defined herein.

The term “S-sulfonamide” describes an —S(═O)₂—NR′R″— end group or a —S(═O)₂—NR′— linking group, as these phrases are defined hereinabove, where R′ and R″ are as defined herein.

The term “phosphonate” describes a —P(═O)(OR′)(OR″) end group or a —P(═O)(OR′)(O)— linking group, as these phrases are defined hereinabove, with R′ and R″ as defined herein.

The term “phosphate” describes an —O—P(═O)₂(OR′) end group or a —O—P(═O)₂(O)— linking group, as these phrases are defined hereinabove, with R′ as defined herein. This term further encompasses the term thiophosphonate.

The term “thiophosphate” describes an —O—P(═O)(═S)(OR′) end group or a —O—P(═O)(═S)(O)— linking group, as these phrases are defined hereinabove, with R′ as defined herein.

The term “carbonyl” or “carbonylate” as used herein, describes a —C(═O)—R′ end group or a —C(═O)— linking group, as these phrases are defined hereinabove, with R′ as defined herein. Alternatively, R′ can be halide, or any other reactive derivative. This term encompasses the term “thiocarbonyl”.

The term “thiocarbonyl” as used herein, describes a —C(═S)—R′ end group or a —C(═S)— linking group, as these phrases are defined hereinabove, with R′ as defined herein.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The term “cyano” describes a —C≡N group.

The term “isocyanate” describes an —N═C═O group.

The term “nitro” describes an —NO₂ group.

The term “azo” describes an —N═NR′ end group or an —N═N— linking group, as these phrases are defined hereinabove, with R′ as defined hereinabove.

The term “carboxylate” describes a —C(═O)—OR′ end group or a —C(═O)—O— linking group, as these phrases are defined hereinabove, where R′ is as defined herein. This term encompasses the terms O-carboxylate, C-thiocarboxylate, and O-thiocarboxylate, as well as various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters.

The term “carbamate” describes an R″OC(═O)—NR′— end group or a —OC(═O)—NR′— linking group, as these phrases are defined hereinabove, with R′ and R″ as defined herein. This term encompasses the terms O-carbamate, thiocarbamate and include various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters.

The term “amide” describes a —C(═O)—NR′R″ end group or a —C(═O)—NR′— linking group, as these phrases are defined hereinabove, where R′ and R″ are as defined herein. This term encompasses the term N-amide.

The term “N-amide” describes a R′C(═O)—NR″— end group or a R′C(═O)—N— linking group, as these phrases are defined hereinabove, where R′ and R″ are as defined herein.

The term “hydrazine” describes a —NR′—NR″R′″ end group or a —NR′—NR″— linking group, as these phrases are defined hereinabove, with R′, R″, and R′″ as defined herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking group, as this phrase is defined hereinabove, connecting two or more moieties at two or more positions thereof. Representative examples are piperidine, piperazine, tetrahydrofurane, tetrahydropyrane, morpholino and the like.

The term “ester” describes a moiety containing a carboxylate group, as defined herein.

An “alkenyl” group describes an alkyl group which consists of at least two carbon atoms and at least one carbon-carbon double bond.

An “alkynyl” group describes an alkyl group which consists of at least two carbon atoms and at least one carbon-carbon triple bond.

A “dienophile” group describes a group which comprises at least two conjugated double-double boned.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-D shows a microfluidic device generated according to the teachings of the present invention and use thereof in a lab-on-chip protein synthesis. (A) PDMS device composed of connected chambers made by standard soft lithography was sealed by a daisy-coated glass cover slip. The coating was restricted to a small area of the cover slip (9 mm in diameter; marked black circle). (B) UV photo-activation of a small region (24 μm in diameter) on the chip was carried out in a single chamber under the microscope. Chemical binding of biotin to the de-protected amines followed by coupling of SA-conjugated genes coding for eGFP under T7 RNA polymerase (dsDNA, 1200 base pairs) was done by continuous flushing of molecules through the inlets. Fluorescence labeling: SA-FITC conjugated to DNA (green), one protein per DNA, as well as direct end-labeling of DNA by Cy5 dye. Scale-bar is 50 μm. (C-D) Kinetics of localized on-chip eGFP synthesis followed by its diffusion outwards was monitored after injecting wheat germ coupled transcription/translation extract into the device. The graph in (C) shows the integrated fluorescence intensity over ˜½ of the total area of the chamber and located at its center.

FIGS. 2A-B are respectively, a schematic diagram and cross-sectional representation of an exemplary device generated according to the teachings of the present invention.

### DESCRIPTION OF THE PREFERRED EMBODIMENTS

The present invention is of microfluidic devices and methods of generating and using same in numerous analytical and synthetic applications.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present inventor has previously described a new approach for single step light directed immobilization of biomolecules onto chips (array technology), see PCT Application No. WO2006/064505.

Whilst reducing the present invention to practice, the present inventor has uncovered a novel approach for photo-directed immobilization of biomolecules to microfluidic devices without compromising the hermetic sealing of the device which is of particular importance in nano-scale reaction volumes. Basically, coating of the microfluidic device with a photoactivatable compound that comprises the photoactivatable moiety for biomolecule immobilization, is restricted to a region (e.g., center) of the microfluidic device which allows sealing of the chip tightly at its periphery while retaining photo-activity of the compound in the center.

As is illustrated hereinbelow and in the Examples section which follows, the present inventor was able to immobilize genes coding for egfp under T7 RNA polymerase in a small region of a microfluidic device using UV-photolithography at the center of a ˜5 nanoliter reaction chamber. UV-photolithography, chemical binding of biotin to the de-protected amines and immobilization of SA-DNA were carried out inside the sealed chambers with the device mounted on a microscope and reagents injected under hydrostatic pressure through thin tubing. Cell-free transcription reagents were flowed into the reaction site to synthesize eGFP from the localized genes.

Microfluidic devices generated according to the teachings of the present invention can be employed in a myriad of microfluidic applications including various chemical and biochemical analyses and syntheses, both for preparative and analytical applications.

Thus, according to one aspect of the present invention there is provided a microfluidic device. The microfluidic device comprising a substrate having formed therein microfluidic paths, at least a portion of the microfluidic paths having attached thereto a plurality of monolayers, wherein at least a portion of the monolayers comprises a photoactivatable group capable of generating a reactive group upon exposure to a light source, the reactive group being for binding a screenable moiety.

As used herein the phrase “microfluidic device” refers to an analytical or synthetic device in which nanoscale and mesoscale volumes of fluids are flowed. Microfluidic devices of the present invention can be incorporated in complicated systems such as described hereinbelow.

As used herein a “microfluidic path” refers to a flow channel or chamber of a micrometric size (e.g., 0.01 to 100 μm in diameter) through which a solution can flow (e.g., reaction mixture or vapor). Typically the microfluidic path is formed from a (multiplexed) flow layer [in which the micro fluidic channel is carved such as by etching (preferably made of an elastomeric material or silica-based substrate)] and a cover layer (e.g., glass or plastics) sealed thereto, such that the cover layer forms one wall of the microfluidic path. Alternatively, the device once removed from the mother mold is sealed to a thin elastomeric membrane such that the flow path is totally enclosed in elastomeric material. The resulting elastomeric device can then optionally be joined to a substrate support. Preferably, at least portion of the microfluidic paths of the device of the present invention are in fluid communication.

As used herein the term “substrate” refers to any one or more materials or a combination of materials from which a micripfluidic device can be formed. Preferably, the substrate material is substantially non-fluorescent or emits light of a wavelength range that does not interfere with the photoactivation. Examples of such materias include, but are not limited to, silica-based materials (examplified hereinbelow) and elastomeric materials.

The term “elastomer” and “elastomeric” as used herein refers to the general meaning as used in the art. Thus, for example, Allcock et al. (Contemporary Polymer Chemistry, 2nd Ed.) describes elastomers in general as polymers existing at a temperature between their glass transition temperature and liquefaction temperature. Elastomeric materials exhibit elastic properties because the polymer chains readily undergo torsional motion to permit uncoiling of the backbone chains in response to a force, with the backbone chains recoiling to assume the prior shape in the absence of the force. In general, elastomers deform when force is applied, but then return to their original shape when the force is removed. The elasticity exhibited by elastomeric materials can be characterized by a Young's modulus. The elastomeric materials utilized in the microfluidic devices disclosed herein typically have a Young's modulus of between about 1 Pa-1 TPa, in other instances between about 10 Pa-100 GPa, in still other instances between about 20 Pa-1 GPa, in yet other instances between about 50 Pa-10 MPa, and in certain instances between about 100 Pa-1 MPa. Elastomeric materials having a Young's modulus outside of these ranges can also be utilized depending upon the needs of a particular application. Examples of elastomeric materials which can be used to fabricate the devices of the present invention include, but are not limited to, GE RTV 615 (formulation), a vinyl-silane crosslinked (type) silicone elastomer (family e.g., PDMS).

The choice of materials typically depends upon the particular material properties (e.g., solvent resistance, stiffness, gas permeability, and/or temperature stability) required for the application being conducted. Additional details regarding the type of materials that can be used in the manufacture of the components of the microfluidic devices disclosed herein are set forth in Unger et al. (2000) Science 288:113-116, and PCT Publications WO 02/43615, and WO 01/01025. Exemplary low-background substrates include those disclosed by Cassin et al., U.S. Pat. No. 5,910,287 and Pham et al., U.S. Pat. No. 6,063,338.

As mentioned herein above, at least a portion of the microfluidic paths is coated with a polymeric monolayer.

According to a preferred embodiment of this aspect of the present invention,

the coat is composed of a compound which can be represented by the general formula I below:

X-L—Y Formula I

wherein X is the functionalized group capable of binding to a substrate; L is the polymer capable of forming a monolayer on a substrate; and Y is a photoactivatable group capable of generating a reactive group upon exposure to light.

The functionalized group is preferably selected such that it binds to the substrate by reacting with at least one functional group present on a surface of a substrate.

Preferred functionalized groups according to the present invention comprise one or more reactive silyl group(s).

As used herein, the phrase “reactive silyl group” describes a residue of a compound comprising at least one silicon atom and at least one reactive group, such as an alkoxy or halide, such that the silyl group is capable of reacting with a functional group, for example on a surface of a substrate, to form a covalent bond with the surface. For example, the reactive silyl group can react with the surface of a silica substrate comprising surface Si—OH groups to create siloxane bonds between the compound and the silica substrate.

Exemplary reactive silyl groups that are usable in the context of the present invention include, without limitation, trialkoxysilanes, alkyldialkoxysilanes, alkoxydialkylsilanes, trihalosilanes, alkyldihalosilanes and dialkylhalosilanes. Such reactive groups are easily reacted when contacted with free hydroxyl groups on a surface of solid surfaces and particularly with such hydroxyl groups on a silica surface.

Herein, the terms “silica” and “SiO₂” are used interchangeably.

In a preferred embodiment of the present invention the reactive silyl group is trialkoxysilane such as, for example trimethoxysilane, triethoxysilane, tripropyloxysilane or trihalosilane such as, for example, trichlorosilane.

The functionalized group according to the present invention may further include a chemical moiety that is terminated with the reactive silyl group. Such a chemical moiety can comprises, for example, alkyl, alkenyl, aryl, cycloalkyl and derivatives thereof, as these terms are defined herein.

Preferably, the functionalized group comprises an alkyl terminating with a trialkoxysilane.

As discussed hereinabove, the polymer is selected so as to form a monolayer on the substrate. Thus, the polymer group in the compounds of the present invention may be any hydrophobic, hydrophilic and amphyphilic polymer that has suitable characteristics for forming a monolayer. Such characteristics include, for example, long, relatively inert chains, which may interact therebetween via e.g., hydrogen or Van-der-Waals interactions.

A preferred polymer according to the present invention comprises polyethylene glycol (PEG). As described hereinabove, PEG is characterized by resistance to nonspecific absorptions of biomolecules and is therefore beneficial for use in some contexts of the present invention. In addition, when self-assembled on a substrate, PEG chains typically interact therebetween via hydrogen bonds, so as to produced a well-ordered monolayered film.

The polyethylene glycol residue in the compounds of the present invention can be derived from PEGs having a molecular weight that ranges from about 400 grams/mol and about 10000 grams/mol. Preferred PEGs are those having a molecular weight that ranges from about 2000 grams/mol and about 5000 grams/mol. Such PEGs allow the productions of a monolayered film when deposited on a solid surface in the presence of a functionalized group, as described hereinabove.

The polyethylene glycol residue may be substituted or unsubstituted and can be represented by the general Formula II below:

—(CR¹R²CR³R⁴O)n- Formula II

wherein n is an integer from 10 to 200; and R¹, R², R³ and R⁴ are each independently selected from the group consisting of hydrogen, alkyl, cycloalkyl, aryl, alkenyl alkynyl, alkoxy, thioalkoxy, aryloxy and thioaryloxy.

In a preferred embodiment, the PEG is unsubstituted such that R¹, R², R³ and R⁴ are each hydrogen.

In another preferred embodiment, the PEG residue is a medium-sized residue such that n is an integer from 60 to 100.

The polymer is preferably attached to the functionalized group described above via a linking moiety.

Exemplary linking moieties include, without limitation, oxygen, sulfur, amine, amide, carboxylate, carbamate, sulphonate, sulphonamide, phosphate, hydrazine, hydrazide, as these terms are defined herein and derivatives thereof.

In a representative example the linking moiety is an amide, formed between a carboxylic end group of the polymer and an amine end group of the functionalized moiety, as is detailed hereinunder.

The compounds of the present invention, by comprising the functionalized group and the polymer described hereinabove, readily form self-assembled monolayers when contacted with a substrate, in a one-step, simple to perform, reaction.

As the polymer residue in the compounds of the present invention further has a photoactivatable group attached thereto, each of the formed monolayers has a photoactivatable group attached thereto.

As used herein, the phrase “photoactivatable group” describes a group that is rendered active when exposed to photoactivation, namely when exposed to light. Photoactivatable groups typically comprise a protected reactive group, which upon exposure to light are de-protected, so as to generate a reactive group.

As used herein, the phrase “reactive group” describes a chemical moiety that is capable of interacting with another moiety. This interaction typically results in a bond formation between these moieties, whereby the bond can be, for example a covalent bond, a hydrogen bond, a coordinative bond, or a ionic bond.

Representative examples of reactive groups include, without limitation, amine, hydroxy, thiohydroxy, halo, alkoxy, thioalkoxy, aryloxy, thioaryloxy, carboxylate, phosphate, phosphonate, sulfate and sulfonate, as these terms are defined herein.

Depending on the intended use of the compound, the photoactivatable group is selected so as to generate a desired reactive group

Thus, for example, a photoactivatable group that comprises a carbamate can generate upon exposure to light amine as the reactive group.

The photoactivatable groups according to the present invention are preferably derived from photoactivatable compounds and therefore preferably include a residue of, for example, photoactivatable compounds that has light-absorbing characteristics such as 6-nitrovertaryl chloroformate, 6-nitrovertaryl carbonyl, 2-nitrotoluene, 2-nitroaniline, phenacyl, phenoxy, azidoaryl, sulfonic ester, desyl, p-hydroxyphenacyl, 7-methoxy coumarin, o-ethylacetophenone, 3,5-dimethylphenacyl, dimethyl dimethoxybenzyloxy carbonyl, 5-bromo-7-nitroindolinyl, o-hydroxy-α-methyl cinnamoyl and 2-oxymethylene anthraquinone.

When exposed to light such as, for example, UV, IR, or visible light or a monochromatic light of a predetermined wavelength, reactive groups, which are capable of binding a desired moiety, preferably a screenable moiety, as is detailed hereinunder, are generated.

The above-described compounds can be readily prepared using a simple two-steps synthesis. A process of preparing the compounds is described in details in PCT Application No. WO2006/064505 to the present inventor.

As mentioned hereinabove, the present inventor has configured a novel approach for producing the microfluidic devices of the present invention.

Thus, according to another aspect of the present invention there is provided a method of manufacturing a microfluidic device.

The method according to this aspect of the present invention comprising:

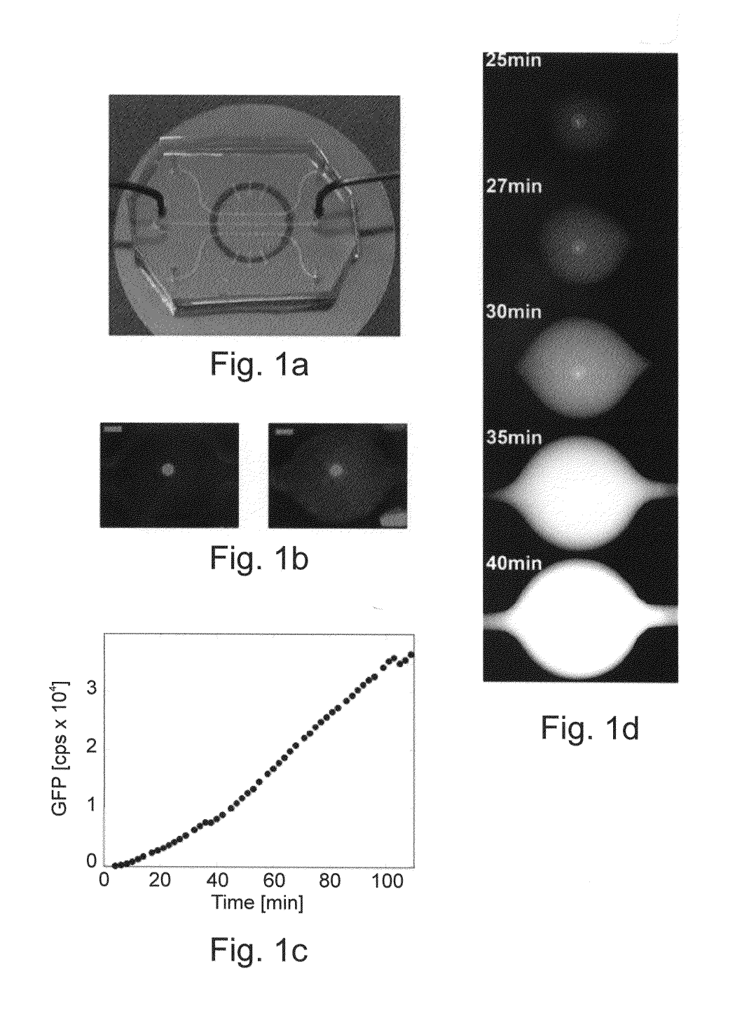

Providing a substrate for generating the microfluidic device. Preferably the cover layer of the device is provided (e.g., flat surface, see FIG. 1a);

Providing a compound having a functionalized group capable of binding to the substrate (e.g., cover layer), a photoactivatable group capable of generating a reactive group upon exposure to a light source, and a polymer capable of forming a monolayer on the substrate, as described hereinabove;

Coating the substrate with the compound to thereby provide a substrate having a monolayer of the polymer attached thereto, wherein the polymer has the photoactivatable group attached thereto. Of note, coating can be effected at any area of the substrate exclusive of the area of the adhesive layer (e.g., center and not periphery) so as to allow sealing of the microfluidic device while retaining functionality of the photoactivatable groups (see Example 1 of the Examples section which follows).

Exposing at least a portion of the center region of the substrate to a light, to thereby generate the reactive group at center of the microfluidic device; and

Binding a screenable moiety to the reactive group.

It will be appreciated that sealing the microfluidic device is preferably effected prior to exposing the device to light and immobilizing the screenable moiety.

In a preferred embodiment, the substrate material is substantially non-reactive with the screenable moiety, thus preventing non-specific binding between the substrate and the moiety or other components of an assay mixture. Methods of coating substrates with materials to prevent non-specific binding are generally known in the art. Exemplary coating agents include, but are not limited to cellulose, bovine serum albumin, and poly(ethyleneglycol). The proper coating agent for a particular application will be apparent to one of skill in the art.

As discussed hereinabove, the substrate and the compound of the present invention are selected such that upon contacting the polymer with the substrate, a self-assembled monolayered film of the polymer forms on the substrate surface, in a one-step reaction.

The contacting procedure is preferably effected by incubating the compound of the present invention with the selected substrate, preferably in the presence of an organic solvent such as, for example, toluene.

Once a monolayered film of the polymer is deposited on the substrate surface, the reactive group for binding a screenable moiety can be generated by exposing a pre-selected area of the substrate to light.

Depending on the selected photoactivatable group and the active wavelength in which it is active, the light can be a UV, IR or visible light, or, optionally and preferably, the light can be a monochromatic light of a predetermined wavelength.

Exposure of a limited area of the substrate to light is preferably effected using a photo mask to illuminate selected regions the substrate and avoid coating the substrate at the periphery. However, other techniques may also be used. For example, the substrate may be translated under a modulated laser or diode light source. Such techniques are discussed in, for example, U.S. Pat. No. 4,719,615 (Feyrer et al.), which is incorporated herein by reference. In alternative embodiments a laser galvanometric scanner is utilized. In other embodiments, the synthesis may take place on or in contact with a conventional liquid crystal (referred to herein as a “light valve”) or fiber optic light sources. By appropriately modulating liquid crystals, light may be selectively controlled so as to permit light to contact selected regions of the substrate. Alternatively, synthesis may take place on the end of a series of optical fibers to which light is selectively applied. Other means of controlling the location of light exposure will be apparent to those of skill in the art.

The substrate may be irradiated either in contact or not in contact with a solution and is, preferably, irradiated in contact with a solution. The solution may contain reagents to prevent the by-products formed by irradiation. Such by-products might include, for example, carbon dioxide, nitrosocarbonyl compounds, styrene derivatives, indole derivatives, and products of their photochemical reactions. Alternatively, the solution may contain reagents used to match the index of refraction of the substrate. Reagents added to the solution may further include, for example, acidic or basic buffers, thiols, substituted hydrazines and hydroxylamines, or reducing agents (e.g., NADH).

In an exemplary embodiment, exposing the substrate to light is effected so as to provide a patterned substrate in which reactive groups are generated according to a pre-selected pattern. The pattern can be printed directly onto the substrate or, alternatively, a “lift off” technique can be utilized. In the lift off technique, a patterned resist is laid onto the substrate or onto the light source. Resists are known to those of skill in the art. See, for example, Kleinfield et al., J. Neurosci. 8:4098-120 (1998). In some embodiments, following removal of the resist, a second pattern is printed onto the substrate on those areas initially covered by the resist; a process that can be repeated any selected number of times with different components to produce an array having a desired format.

Once the reactive group is generated the device is preferably sealed using methods which are well known in the art. Low fluorescence adhesives which provide sealing and cover constructions are preferably used. Such adhesives are dimensionally stable and do not flow into microfluidic channels. They adhere to the cover layer without creating voids or gaps that may allow migration of components from one path to adjacent path, and they exhibit good stability to moisture and temperature change. Adhesives used in accordance with the present invention can be either flexible or rigid, but should preferably be clear and colorless (such adhesives can be obtained from Adhesives Research Inc.). Other adhesives include, but are not limited to, pressure sensitive adhesives, such as ethylene-containing polymers, urethane polymers, butyl rubber, butadiene-acrylonitrile polymers, butadiene-acrylonitrile-isoprene polymers, and the like. See, for example, U.S. Pat. No. 5,908,695 and references cited therein. As shown in Example 1 of the Examples section which follows, binding of egfp coding sequence was effected following sealing.

Binding the screenable moiety can be effected by directly attaching the moiety to the reactive group.

Alternatively, binding the screenable moiety is effected via a mediating moiety. As used herein, the phrase “mediating moiety” describes a mediating agent or a plurality of mediating agents being linked therebetween that may bind to both the reactive group and the screenable moiety and thus mediate the binding of the screenable moiety to the reactive group.

The mediating moiety can thus be a bifunctional moiety, having two reactive groups, each independently capable of reacting with the reactive group attached to the substrate or the screenable moiety. Alternatively, the mediating moiety can comprise two or more moieties, whereby the first moiety can be attached to the reactive group and to a second mediating moiety, whereby the second mediating moiety can bind the screenable moiety.

Optionally and preferably, the mediating moiety comprises an affinity pair, such as, for example, the biotin-avidin affinity pair. The biotin-avidin affinity pair highly useful for integrating biomolecules on the substrate.

Alternatively, the mediating moiety can comprise biotin. When attached to the reactive group, biotin can bind a variety of chemical and biological substances that are capable of reacting with the free carboxylic group thereof.

As used herein the phrase “screenable moiety” refers to biological moieties or chemical moieties. Such a biological or chemical moiety can be, for example, a drug or a drug target. A chemical moiety that functions as a drug for example, can be a small organic molecule, which can, for example, inhibit or activate a process. A screenable moiety that functions as a drug can be, for example, a growth factor or hormone. Representative examples of biological moieties that are suitable for use in the context of the present invention include, without limitation, peptides, proteins, glycoproteins, proteoglycans, nucleic acids, polynucleotides, oligonucleotides, antibodies, carbohydrates, lipids, cells, microorganisms, enzymes, hormones, steroids, second messengers and growth factors.

Devices of the present invention are generally constructed utilizing single and multilayer soft lithography (MSL) techniques and/or sacrificial-layer encapsulation methods. The basic MSL approach involves casting a series of elastomeric layers on a micro-machined mold, removing the layers from the mold and then fusing the layers together. In the sacrificial-layer encapsulation approach, patterns of photoresist are deposited wherever a channel is desired. These techniques and their use in producing microfluidic devices is discussed in detail, for example, by Unger et al. (2000) Science 288:113-116, by Chou, et al. (2000) “Integrated Elastomer Fluidic Lab-on-a-chip-Surface Patterning and DNA Diagnostics, in Proceedings of the Solid State Actuator and Sensor Workshop, Hilton Head, S.C.; and in PCT Publication WO 01/01025

Devices of the present invention can comprise numerous microfluidic paths (e.g., up to tens of thousands) and can achieve high reaction site densities (e.g., over 1000-4000 reaction sites/cm²). The large number of reaction sites and densities that can be obtained is also a consequence of the ability to fabricate very small wells or cavities. For example, reaction sites (chambers) typically have a volume of less than 50 mL; in other instances less than 40 mL, 30 mL, 20 mL or 10 mL; and in still other instances less than 5 mL or 1 mL. As a specific example, certain devices have reaction chambers that are 300 microns long, 300 microns wide and 10 microns deep.

Systems which comprise the device of the present invention may comprise a variety of different detection modalities at essentially any location on the microfluidic device. Detection can be achieved using detectors that are incorporated into the device or that are separate from the device but aligned with the region of the device to be detected.

A number of different detection strategies can be utilized with the microfluidic devices that are provided herein. Selection of the appropriate system is informed in part on the type of event and/or agent being detected. The detectors can be designed to detect a number of different signal types including, but not limited to, signals from radioisotopes, fluorophores, chromophores, electron dense particles, magnetic particles, spin labels, molecules that emit chemiluminescence, electrochemically active molecules, enzymes, cofactors, enzymes linked to nucleic acid probes and enzyme substrates.

Illustrative detection methodologies suitable for use with the present microfluidic devices include, but are not limited to, light scattering, multichannel fluorescence detection, UV and visible wavelength absorption, luminescence, differential reflectivity, and confocal laser scanning. Additional detection methods that can be used in certain application include scintillation proximity assay techniques, radiochemical detection, fluorescence polarization, fluorescence correlation spectroscopy (FCS), time-resolved energy transfer (TRET), fluorescence resonance energy transfer (FRET) and variations such as bioluminescence resonance energy transfer (BRET). Additional detection options include electrical resistance, resistivity, impedance, and voltage sensing.

Detection occurs at a “detection section,” or “detection region”, namely at the reaction site. The detection section can be in communication with one or more microscopes, diodes, light stimulating devices (e.g., lasers), photomultiplier tubes, processors and combinations of the foregoing, which cooperate to detect a signal associated with a particular event and/or agent. Often the signal being detected is an optical signal that is detected in the detection section by an optical detector. The optical detector can include one or more photodiodes (e.g., avalanche photodiodes), a fiber-optic light guide leading, for example, to a photomultiplier tube, a microscope, and/or a video camera (e.g., a CCD camera).

Detectors can be microfabricated within the microfluidic device, or can be a separate element. If the detector exists as a separate element and the microfluidic device includes a plurality of detection sections, detection can occur within a single detection section at any given moment. Alternatively, scanning systems can be used. For instance, certain automated systems scan the light source relative to the microfluidic device; other systems scan the emitted light over a detector, or include a multichannel detector. As a specific illustrative example, the microfluidic device can be attached to a translatable stage and scanned under a microscope objective. A signal so acquired is then routed to a processor for signal interpretation and processing. Arrays of photomultiplier tubes can also be utilized. Additionally, optical systems that have the capability of collecting signals from all the different detection sections simultaneously while determining the signal from each section can be utilized.

The detector can include a light source for stimulating a reporter that generates a detectable signal. The type of light source utilized depends in part on the nature of the reporter being activated. Suitable light sources include, but are not limited to, lasers, laser diodes and high intensity lamps. If a laser is utilized, the laser can be utilized to scan across a set of detection sections or a single detection section. Laser diodes can be microfabricated into the microfluidic device itself. Alternatively, laser diodes can be fabricated into another device that is placed adjacent to the microfluidic device being utilized to conduct a thermal cycling reaction such that the laser light from the diode is directed into the detection section.

Detection can involve a number of non-optical approaches as well. For example, the detector can also include, for example, a temperature sensor, a conductivity sensor, a potentiometric sensor (e.g., pH electrode) and/or an amperometric sensor (e.g., to monitor oxidation and reduction reactions). A number of commercially-available external detectors can be utilized. Many of these are fluorescent detectors because of the ease in preparing fluorescently labeled reagents. Specific examples of detectors that are available include, but are not limited to, Applied Precision ArrayWoRx (Applied Precision, Issaquah, Wash.)).

Various number of microfluidic devices can be generated according to the teachings of the present invention.

The following describes a simple configuration of the microfluidic devices of the present invention, which can be utilized to conduct a variety of analyses. It should be understood that this configuration is exemplary and that modifications thereof will be apparent to those skilled in the art.

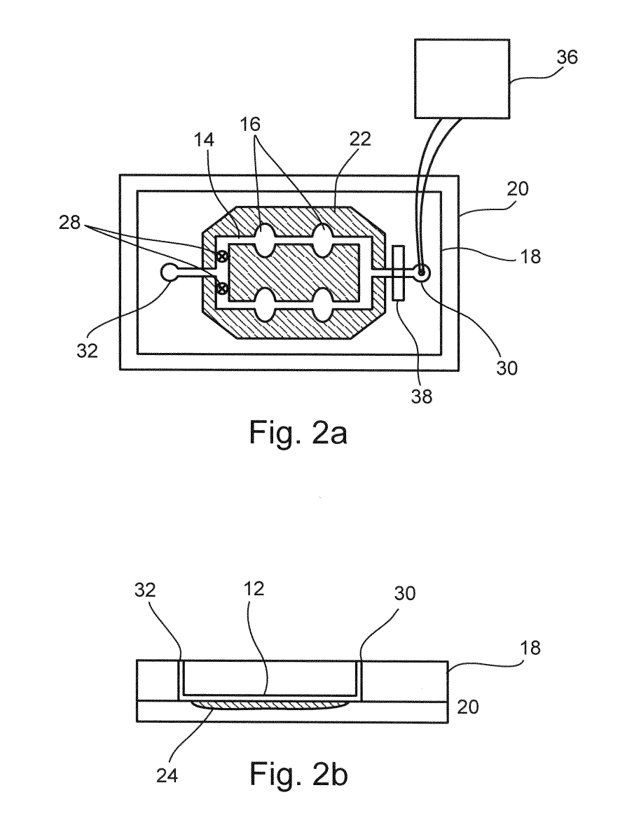

Thus, FIGS. 2a-b are schematic illustrations of a top view (FIG. 2a) and a cross-sectional view (FIG. 2b) of a microfluidic device 10, according to various exemplary embodiments of the present invention. In a simple configuration, microfluidic device 10 comprises a number of microfluidic paths 12 forming microfluidic channels 14 and microfluidic chambers 16. Microfluidic paths 12 are formed in a flow layer 18 which is preferably covered by solid cover layer 20. A selected region 22 of cover layer 20 is coated with compound 24 of the present invention. The type of attachment of flow layer 18 to cover layer 20 depends on the materials from which flow layer 18 and cover layer 20 are fabricated. The preferred configuration is elastomeric flow layer and glass or quartz cover layer in which flow layer 18 can be easily attached to cover layer 20 negating the need for introducing a foreign adhesive layer (just treating the elastomeric material with plasma oxidation). Yet, this need not necessarily be the case, since, for some applications, it may be desired to use other materials for flow layer 18 and/or cover layer 20. Thus, in an alternative embodiment, device 10 comprises an adhesive layer (not shown) disposed between flow layer 18 to cover layer 20 which guarantees their attachment.

According to a preferred embodiment of the present invention device 10 further comprises valves 28 which may be operated using any mode of operation known in the art. For example, a second layer of intersecting guard channels (not shown) formed of, e.g., an elastomeric material may be disposed over flow layer 18 such that valves 28 are formed by intersection of channels separated by a membrane (e.g., elastomeric membrane) that can be deflected into or retracted from the flow channel in response to an actuation force. Valves 28 can be used for directing flow and/or isolating the reaction site when sufficient pressure is applied. Device 10 can also comprise an inlet port 30 and an outlet port 32 at least one of which being in fluid communication with an external reagent reservoir 36 such as by tubing (such as for infusing the screenable moiety). Fluids may be passively or actively infused into the flow channels such as by capillary forces or pump 38 (e.g., external pumps, e.g., peristaltic pumps or electro-osmotically pumps).

The aforedescribed devices may be utilized in temperature controlled reactions (e.g., thermocycling reactions). In such a case, the resulting device is preferably placed in a temperature control plate (not shown), for example, to control the temperature at the various reaction sites. In the case of thermocycling reactions, the device can be placed on any of a number of thermocycling plates.

More complex configurations include matrix configurations such as those described in U.S. Pat. Application No. 2005/0084421.

The following describes various applications for the devices of the present invention.

Devices with the ability to conduct nucleic acid amplifications (such as described above) would have diverse utilities. For example, such devices could be used as an analytical tool to determine whether a particular target nucleic acid of interest is present or absent in a sample. Thus, the devices could be utilized to test for the presence of particular pathogens (e.g., viruses, bacteria or fungi), and for identification purposes (e.g., paternity and forensic applications) wherein a primer pair of interest coats the substrate cover layer as described above. Such devices could also be utilized to detect or characterize specific nucleic acids previously correlated with particular diseases or genetic disorders (e.g., diagnostic applications). When used as analytical tools, the devices could also be utilized to conduct genotyping analyses and gene expression analyses (e.g., differential gene expression studies). Alternatively, the devices can be used in a preparative fashion to amplify sufficient nucleic acid for further analysis such as sequencing of amplified product, cell-typing, DNA fingerprinting and the like. Amplified products can also be used in various genetic engineering applications, such as insertion into a vector that can then be used to transform cells for the production of a desired protein product.

While useful for conducting a wide variety of nucleic acid analyses, the devices of the present invention can also be utilized in a number of other applications as well. The devices can be utilized to analyze essentially any interaction between two or more species that generates a detectable signal or a reaction product that can reacted with a detection reagent that generates a signal upon interaction with the reaction product.

Thus, for example, the devices can be utilized in a number of screening applications to identify test agents that have a particular desired activity. As a specific example, the devices can be utilized to screen compounds for activity as a substrate or inhibitor of one or more enzymes. In such analyses, test compound and other necessary enzymatic assay reagents (e.g., buffer, metal ions, cofactors and substrates) are introduced in the reaction site. The enzyme may have been previously immobilized according to the above teachings. Reaction (if the test compound is a substrate) or inhibition of the reaction (if the test compound is an inhibitor) is detected. Such reactions or inhibition can be accomplished by standard techniques, such as directly or indirectly monitoring the loss of substrate and/or appearance of product.

Devices with sufficiently large flow paths (micrometer range) can also be utilized to conduct cellular assays to detect interaction between a cell and one or more reagents. For instance, certain analyses involve determination of whether a particular cell type is present in a sample. For example, test compounds can be screened for ability to trigger or inhibit a cellular response, such as a signal transduction pathway of an immobilized cell of interest. In such an analysis, test compound is introduced once the cell has been immobilized in an appropriate buffer environment. The reaction site is then checked to detect formation of the cellular response.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Photolithography of Biomolecules and Protein Biosynthesis within Microfluidic Channels

A microfluidic (MICROFLUIDIC) device made of poly-di-methyl-siloxane (PDMS) elastomer by standard soft lithography [D. C. Duffy, J. C. McDonald, O. J. A. Schueller, G. M. Whitesides, Analytical Chemistry 1998, 70, 4974] was sealed with a daisy-coated glass cover slip. The coating of daisy was restricted to a small area of the cover slip (FIG. 1A), which allowed sealing the chip tightly with the PDMS at its periphery while retaining the photo-activity of daisy in the center.

Genes coding for egfp under T7 RNA polymerase (1200 bp dsDNA) were immobilized to a small region (24 μm in diameter) using UV-photolithography at the center of a ˜5 nanoliter reaction chamber (FIG. 1B). UV-photolithography, chemical binding of biotin to the de-protected amines and immobilization of SA-DNA were carried out inside the sealed chambers with the device mounted on a microscope and reagents injected under hydrostatic pressure through thin tubing. The transcription/translation reaction was then introduced into the device to synthesize eGFP from the localized genes. Synthesis of eGFP was imaged at a concentration above a ˜5 nM. FIGS. 1C-D show time laps images and continuous kinetics of on-chip eGFP expression followed by its diffusion.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. A microfluidic device comprising a substrate having formed therein microfluidic paths, at least a portion of said microfluidic paths having attached thereto a plurality of monolayers, wherein at least a portion of said monolayers comprises a photoactivatable group capable of generating a reactive group upon exposure to a light source, said reactive group being for binding a screenable moiety.

2. The microfluidic device of claim 1, wherein said monolayers being composed of a compound which comprises a general formula I:
X-L—Y Formula I
wherein:
X is a functionalized group capable of binding to said substrate;
L is a polymer capable of forming said monolayer onto said substrate; and
Y is said photoactivatable group capable of generating said reactive group upon exposure to said light.

3. The microfluidic device of claim 2, wherein said functionalized group comprises at least one reactive silyl group.

4. The microfluidic device of claim 3, wherein said reactive silyl group is selected from the group consisting of trialkoxysilane, alkyldialkoxysilane, alkoxydialkylsilane, trihalosilane, alkyldihalosilane and dialkylhalosilane.

5. The microfluidic device of claim 4, wherein said reactive silyl group is trialkoxysilane.

6. The microfluidic device of claim 5, wherein said functionalized group comprises an alkyl terminating with said trialkoxysilane.

7. The microfluidic device of claim 2, wherein said functionalized group comprises at least one reactive silyl group selected from the group consisting of trialkoxysilane, alkyldialkoxysilane, alkoxydialkylsilane, trihalosilane, alkyldihalosilane and dialkylhalosilane.

8. The microfluidic device of claim 1, wherein said polymer comprises a substituted or unsubstituted polyethylene glycol (PEG).

9. The microfluidic device of claim 8, wherein said polyethylene glycol has a molecular weight that ranges from about 400 grams/mol and about 10000 grams/mol.

10. The microfluidic device of claim 9, wherein said polyethylene glycol has a molecular weight that ranges from about 2000 grams/mol to about 5000 grams/mol.

11. The microfluidic device of claim 8, wherein said polyethylene glycol has a general formula II:
—(CR¹R²CR³R⁴O)n- Formula II
wherein:
n is an integer from 10 to 200; and
R¹, R², R³ and R⁴ are each independently selected from the group consisting of hydrogen, alkyl, cycloalkyl, aryl, alkenyl alkynyl, alkoxy, thioalkoxy, aryloxy and thioaryloxy.

12. The microfluidic device of claim 11, wherein R¹, R², R³ and R⁴ are each hydrogen.

13. The microfluidic device of claim 11, wherein n is an integer from 60 to 100.

14. The microfluidic device of claim 2, wherein said functionalized group is attached to said polymer via a linking moiety.

15. The microfluidic device of claim 14, wherein said linking moiety is selected from the group consisting of oxygen, sulfur, amine, amide, carboxylate, carbamate, N-carbamate, sulphonate, sulphonamide, phosphate, hydrazine, hydrazide and derivatives thereof.

16. The microfluidic device of claim 15, wherein said linking moiety is amide.

17. The microfluidic device of claim 1, wherein said reactive group generated upon exposure of said photoactivatable group to said light is selected from the group consisting of amine, hydroxy, thiohydroxy, halo, alkoxy, thioalkoxy, aryloxy, thioaryloxy, carboxylate, phosphate, phosphonate, sulfate and sulfonate.

18. The microfluidic device of claim 1, wherein said photoactivatable group comprises a carbamate.

19. The microfluidic device of claim 18, wherein said reactive group generated upon exposure of said photoactivatable group to said light is amine.

20. The microfluidic device of claim 18, wherein said photoactivatable group comprises a 6-nitrovertaryl chloroformate residue, a 2-nitrobenzyl residue, a 2-nitroanilino residue, a phenacyl residue, a phenoxy residue, an azidoaryl residue, a sulfonic ester residue, a desyl residue, a p-hydroxyphenacyl residue, a 7-methoxy coumarin residue, a o-ethylacetophenone residue, a 3,5-dimethylphenacyl residue, a dimethyl dimethoxybenzyloxy residue, a 5-bromo-7-nitroindolinyl residue, a o-hydroxy-α-methyl cinnamoyl residue and a 2-oxymethylene anthraquinone residue.

21. The microfluidic device of claim 1, wherein said light is selected from the group consisting of UV, IR, visible light and monochromatic light of a predetermined wavelength.

22. The microfluidic device of claim 1, wherein said substrate comprises a silica-based material.

23. The microfluidic device of claim 1, wherein said substrate comprises an elsatomeric material.

24. The microfluidic device of claim 1, wherein said screenable moiety is selected from the group consisting of a screenable moiety and chemical moiety.

25. The microfluidic device of claim 1, wherein said screenable moiety is selected from the group consisting of a peptide, a protein, a glycoprotein, a proteoglycan, a nucleic acid, an oligonucleotide, an antibody, a carbohydrate, a hormone, a steroid, a lipid, a cell, a microorganism, an enzyme, and a growth factor.

26. The microfluidic device of claim 1, wherein said chemical moiety is selected from the group consisting of a chelating agent, an inhibitor, a substrate and a ligand.

27. The microfluidic device of claim 1, further comprising at least one valve for regulating fluid infusion into said microfluidic paths.

28. The microfluidic device of claim 1, wherein said microfluidic paths comprise microchannels and/or microchambers.

29. The microfluidic device of claim 28, wherein said microchambers form reaction sites.

30. The microfluidic device of claim 1, further comprising at least one reagent inlet port.

31. The microfluidic device of claim 1, further comprising at least one reaction outlet port.

32. The microfluidic device of claim 31, further comprising at least one external reservoir being in fluid communication with said reagent inlet port.

33. A method of manufacturing a microfluidic device, the method comprising:
(a) providing a substrate having formed therein microfluidic paths; and
(b) coating said substrate with a compound having a functionalized group bound to said substrate, a photoactivatable group capable of generating a reactive group upon exposure to a light source, said reactive group being for binding a screenable moiety, and a polymer capable of forming a monolayer on said substrate and having said functionalized group and said photoactivatable group attached thereto, so as to form a plurality of monolayers of said polymer over said substrate, wherein said coating is restricted to an area so as to allow sealing of the microfluidic device while retaining functionality of said photoactivatable group.

34. The method of claim 33, further comprising sealing the microfluidic device following step (b).

35. A method of nucleic acid amplification, the method comprising:
(a) providing the microfluidic device of claim 1;
(b) binding at least one nucleic acid template to said reactive group; and
(c) flowing at least one amplification reagent into reaction sites of said microfluidic device under conditions which allow nucleic acid amplification.

36. The method of claim 35, wherein said at least one amplification reagent is selected from the group consisting of primers, polymerase, nucleotides, metal ions, buffer and cofactors.

37. The method of claim 35, wherein the nucleic acid amplification is effected according to a method selected from the group consisting of a PCR, a ligase amplification reaction (LCR), a transcription amplification, a self-sustained sequence replication and a nucleic acid based sequence amplification (NASBA).

38. The method of claim 35, further comprising detecting the amplified product of (c).

39. A method of detecting a nucleic acid sequence of interest, the method comprising:
(a) providing the microfluidic device of claim 1;
(b) binding at, least one nucleic acid probe to said reactive group; and
(c) flowing a labeled probe into reaction sites of said microfluidic device under conditions under conditions allowing detection of the nucleic acid sequence of interest.

40. The method of claim 39, wherein said nucleic acid sequence of interest comprises a SNP.

41. A method of identifying an agent having a desired activity, the method comprising:
(a) providing the microfluidic device of claim 1;
(b) binding a plurality of agents to said reactive groups such that one agent is disposed in one reaction site at the plurality of areaction sites of the microfluidic device; and
(c) flowing reaction mixtures into reaction sites of said microfluidic device;
(d) monitofing the desired activity following said flowing thereby to thereby identify the agent having the desired activity.
